(19) United States
(12) Patent Application Publication
(10) Pub. No.: US 2007/0224301 A1
(43) Pub. Date: Sep. 27, 2007

(54) Compounds from an extract of Artemisia and methods for treating disorders

(76) Inventors: David M. Ribnicky, North Branch, NJ (US); Sithes Logendra, Piscataway, NJ (US); Alexander Poulev, Highland Park, NJ (US); William T. Cefalu, Baton Rouge, LA (US); Ilya Raskin, Manalapan, NJ (US)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 11/385,524

(22) Filed: Mar. 21, 2006

(51) Int. Cl. A61K 36/282; A61K 31/7048; A61K 31/22

(52) U.S. Cl. 424/740; 514/27; 514/548

(57) ABSTRACT

This invention comprises an extract of Artemisia dracunculus that can be used for the treatment and prevention of diabetes, diabetic complications, metabolic syndrome and other comorbidities that share the underlying commonality of insulin resistance. The invention includes the identity of six compounds from the extract that contribute to the activity of the extract by inhibiting protein tyrosine phosphatase-1B (PTP-1B) activity, phosphoenolpyruvate carboxykinase (PEPCK) gene expression or aldose reductase activity (ALR2). The compounds include 4,5-Di-O-caffeoylquinic acid, davidigenin, 6-demethoxycapillarisin, 2′,4-dihydroxy-4′-methoxydihydrochalcone, 2′,4′-dihydroxy-4-methoxdihydrochalcone and sakuranetin.

### BACKGROUND OF THE INVENTION

1. Field of the Invention

The present invention relates to materials and methods for treating a disorder sing plants. More specifically, the invention relates to materials and methods for reating a disorder, such as diabetes, using compounds isolated from an extract of the plant genus Artemisia.

2. Description of Related Art

Diabetes is a complex condition or disease that is most commonly defined by elevated concentrations of blood glucose, with the disorder affecting the metabolism of carbohydrates, fats and proteins. The disorder results from an inability to control blood glucose levels, for example, due to insufficient levels or activity of insulin. Elevated glucose levels, in turn, often lead to secondary health problems that require additional medical treatment. Some of the leading diabetes-related health risks include hyperglycemia, arteriosclerosis, diabetic retinopathy (possibly leading to blindness), cataracts, nephropathy, increased risk of infections, hypertension, nerve disease, risk of amputations, impotence, diabetic ketoacidosis, and dementia. While these health risks are associated with diabetes, they are not, by themselves, useful indicators of diabetes. For example, hypertension may occur with or without diabetes (e.g., due to a genetic predisposition or a high-salt diet).

There are two primary types of diabetes, with many variations of each. Type 1 diabetes generally occurs in childhood and results from the body's inability to produce insulin. Type 2 diabetes is the more prevalent form and results from either insulin deficiency or, more commonly, from insulin resistance.

Insulin resistance is a key pathophysiologic feature of the “metabolic syndrome” and is strongly associated with co-existing cardiovascular risk factors and accelerated atherosclerosis (Haffner S. M., The insulin resistance syndrome revisited, Diabetes Care 19:275-277 (1996)). Due to the clinical consequences associated with insulin resistance in subjects with metabolic syndrome and type 2 diabetes, clinical regimens directed at increasing insulin sensitivity in vivo remain one of the most desirable goals of treatment. Although it is well established that lifestyle modification can improve insulin resistance and effectively improve many of the risk factors associated with the metabolic syndrome, the success of maintaining lifestyle changes in humans over a chronic period is poor. Therefore, strategies to improve insulin resistance by pharmacological means have represented the traditional approach for clinical medicine (Davidson, M. B., Diabetes Mellitus: diagnosis and treatment 4^{th} edition, W. B. Saunders Company, Philadelphia (1998)).

In addition to conventional treatments relying on insulin injections or over-the-counter medications, natural products, including plant materials, have been tried as alternative treatments of conditions such as diabetes. This is perhaps unsurprising, given the great variety of plants in the world. As traditional medicines, plants have been used for a variety of real or imagined ailments, with the same plants frequently being used to “treat” unrelated conditions. One of the many plant families used in traditional herbal remedies is the Artemisia family, with over 400 different species.

One species of Artemisia, Artemisia dracunculus, Yazdanparast et al., Biomedical Letters 5937-141 (1999) has been reported to yield alcohol-based extracts that exhibit an antihyperlipidemic effect on rats fed high-fat diets. The authors of this study did not, however, test for the presence of mutagens or toxins and did not explore the use of such extracts to treat disorders, disease or conditions other than hyperlipidemia. Artemisia dracunulus and other Artemisia species, Artemisia herba-alba, have been reportedly used to treat headaches and dizziness, e.g., in Middle Eastern cultures. (Al-Waili et al., Clinical and Experimental Pharmacology and Physiology 13-569-573 (1986)). Additionally, Swanston-Flatt et al., Proc. Nutr. Soc. 50:641-651 (1991) disclosed the use of tarragon mixtures in treating diabetes, referencing Swanston-Flatt et al., Acta Diabetol. Lat. 26:51-55 (1989), for an explanation that treatments were prepared by mixing homogenized plant material into standard diets. Swanston-Flatt et al. (1991) reported that tarragon, while reportedly shown to reduce body weight, polydipsia and hyperphagia, did not significantly lower blood glucose concentrations. The authors of this study never prepared extracts from the tarragon.

Artemisia pallens was used as a folk remedy for diabetes in southern India and alcoholic extracts of this species were shown to lower blood glucose concentrations in glucose loaded normal rats and in chemically induced diabetic rats (Subramonium et al., Effects of Artemisia pallens Wall. on blood glucose levels in normal and alloxan-induced diabetic rats, J. Ethnopharmacol. 50(1):13-17 (1996)). In Turkish folk medicine Artemisia santonicum was used for the treatment of diabetes and its ability to lower blood glucose was validated in normal and alloxan-induced diabetic rabbits (Korkmaz et al., Effect of Artenzisia santonicum L. on blood glucose in normal and alloxan-induced diabetic rabbits, Phyto Res. 16:675-676 (2002)).

Considerable controversy exists regarding the effect of botanical supplements on the metabolic syndrome in large part because efficacy data for many of the supplements used for this purpose consists of only uncontrolled studies and anecdotal reports.

U.S. Pat. No. 6893,627, issued to inventors of this application, described that an ethanolic extract of Artemisia dracunculus has anti diabetic properties. The in vitro and in vivo studies suggest that an alcoholic extract of Russian Tarragon (Artemisia dracunculus L) may increase insulin action in vivo and several intracellular pathways were identified that may explain the effect. The extract lowered blood glucose levels in both chemically induced diabetic mice lacking insulin and in genetically diabetic mice with insulin resistance. The extract also enhanced insulin stimulated glucose uptake and increased the accumulation of insulin receptor substrate-2 (IRS-2) in skeletal muscle cell cultures of obese rats. The extract was shown to reduce blood insulin levels in mildly diabetic patients (Ribnicky et al., The development of an extract of Artemisia dracunculus for decreasing the insulin resistance associated with diabetes, from concept to clinic. Gordon Research Conference on Agricultural Sciences “Adding more value to production agriculture”, Feb. 13-18, 2005, Ventura, Calif., USA (2005)). The extract was also shown to be safe and non-toxic (Ribnicky et al., Toxicological Evaluation of the Ethanolic Extract of Artemisia dracunculus L. for Use as a Dietary Supplement and in Functional Foods, Food Chem. Tox. 42(4):585-598 (2004)).

Insulin resistance is a key underlying factor for metabolic syndrome and type 2 diabetes. While the precise cause of insulin resistance is not clearly understood, it appears that many downstream signals from the binding of insulin to its receptor are altered as a result of insulin resistance. Protein tyrosine phosphatase-1B (PTP-1B) is a member of the protein tyrosine phosphatase family of enzymes that is localized to the endoplasmic reticulum and dephosphorylates the tyrosine residues of the insulin receptor (Liu G., Protein tyrosine phosphatase 1B inhibition: opportunities and challenges. Current Medicinal Chemistry 10:1407-1421 (2003)). Overexpression studies have shown that PTP-1B dephosphorylates the insulin receptor in vitro leading to an increase in insulin resistance. PTB-1B overexpression also promotes the downregulation of insulin receptor substrate-1 (IRS-1) and insulin-stimulated phosphatidylinositol 3-kinase (PI3-K) activity, also associated with insulin resistance (Venable et al., Overexpression of protein-tyrosine phosphatase-1B in adipocytes inhibits insulin-stimulated phosphoinositide 3-kinase activity without altering glucose transport or Akt/protein kinase B activation, J. Bio. Chem. 275(24):18318-18326 (2000)); Egawa et al., Protein-tyrosine phospahatase-1B negatively regulates insulin signaling in L6 myocytes and Fao hepatoma cells, J. Biol. Chem. 276(13):10207-10211 (2001)). In addition, high tissue levels of PTP-1B have been reported in insulin-resistant diabetic humans as well as insulin resistant diabetic animals (Ahmad et al., Alterations in skeletal muscle protein-tyrosine phosphatase activity and expression in insulin-resistant human obesity and diabetes, J. Clin. Invest. 100(2):449-458 (1997)). Conversely, increased insulin sensitivity and resistance to obesity was observed in animals in which the PTP-1B gene was genetically inactivated (Elchebly et al., Increased insulin sensitivity and obesity resistance in mice lacking the protein tyrosine phosphatase-1B gene, Sci. 283:1544-1548 (1999)). Thus, PTP 1 activity is correlated with increased insulin resistance and a decreased PTP-1B activity is correlated with enhanced insulin sensitivity. PTP-1B inhibition is believed to be promising for the treatment of insulin resistance as well as the co-morbidities of metabolic syndrome associated with insulin resistance (Ukkola et al., Protein phosphatase 1B: a new target for the treatment of obesity and associated co-morbidities, J. Int. Med. 251:467-475 (2002)); Tonks, PTP-1B: from the sidelines to the front lines! FEBS Letters 546:140-148 (2003)).

Insulin resistance is defined as an attenuated biological response to insulin and is manifested in multiple metabolic pathways. Phosphoenolpyruvate carboxykinase (PEPCK) is a rate-controlling enzyme of gluconeogenesis in the liver and plays a key role in the process of glucose homeostasis (Hanson et al., Regulation of phosphoenolpyruvate carboxykinase (GTP) gene expression, Annu. Rev. Biochem. 66:581-611 (1997)). Glucocorticoids and some second messengers, like cAMP, increase the transcription rate of the PEPCK in liver when blood glucose concentrations are low whereas insulin normally represses its transcription when blood glucose levels are high to decrease hepatic glucose output. The inability of insulin to downregulate the transcription of PEPCK allows hepatic glucose output to persist and contributes to the insulin-resistance syndrome common for type 2 diabetes (Valera et al., Transgenic mice overexpressing phosphoenolpyruvate carboxykinase develop non-insulin-dependent diabetes mellitus, Proc. Nat'l. Acad. Sci. USA 91:9151-9154 (1994)). It was shown in Yuan et al., 2002; Chakraborty et al., 2005 that plant-derived drugs and certain plant extracts exert insulin-like effects in hepatocytes by decreasing PEPCK gene expression. U.S. Pat. No. 6,893,627 describes that an extract of Artemisia was shown to decrease PEPCK gene expression in the livers of diabetic animals.

Insulin resistance is the major underlying factor for the development of hyperglycemia and frank diabetes which leads to a multitude of co-morbidities such as diabetic neuropathy, nephropathy, retinopathy and cardiovascular diseases. The enzyme aldose reductase (ALR2), a member of the aldoketo reductase superfamily, is the first enzyme of the polyol pathway and catalyzes the conversion of blood glucose into sorbitol in the presence of nicotinamide adenine dinucleotide phosphate (NADPH) in reduced form. Under normal glucose conditions, ALR2, functions as a scavenging enzyme for toxic aldehydes in nerve cells (Kawamura et al., Aldose reductase: an aldehyde scavenging enzyme in the intraneuronal metabolism of norepinephrine in human sympathetic ganglia, Autonomic Neurosci. 96(2):131-139 (2002)) as well as an enzyme that regulates cell growth (Donohue et al., A delayed-early gene activated by fibroblast growth factor-1 encodes a protein related to aldose reductase, J. Biol. Chem. 269(11):8604-9 (1994); Laeng et al., Long-term induction of an aldose reductase protein by basic fibroblast growth factor in rat astrocytes in vitro, Electrophoresis 16(7):1240-1250 (1995)). Under euglycemic conditions, ALR2 has low affinity for glucose and converts very little glucose into sorbitol. Under hyperglycemic conditions, however, the blood glucose is increased, leading to high glucose concentrations in tissues that have insulin independent glucose entry such as the vascular endothelial cells of peripheral nerves, kidney, and the retina of the eye. This excess glucose is then converted to sorbitol by the ALR2 enzyme. The conversion of sorbitol to fructose by sorbitol dehydrogenase, in contrast, is very slow and, thus, sorbitol accumulates in the cells as it cannot pass through cell membranes. Sorbitol accumulation leads to osmotic stress and damage to the cells. Inhibitors of ALR2 have been shown to block this pathway (De la Fuente et al., 2003; Miyamoto, 2002), by binding to the active site of the enzyme (Wilson et al., A structure of human aldose reductase complexed with the potent inhibitor zopolrestst, Proc. Natl. Acad. Sci. USA 90:9847-9851 (1993); Hohman et al., 1998). Various ALR2 inhibitors have been synthesized (Constantino et al., Synthesis, activity, and molecular modeling of a new series of tricyclic pyridazinones as selective aldose reductase inhibitors, J. Med. Chem. 39:4396-4405 (1996); Severi et al., Synthesis and activity of a new series of chalcones as aldose reductase inhibitors, Eur. J. Med. Chem. 33:859-866 (1996); Lim et al., Synthesis of flavonoids and their effects on aldose reductase and sorbitol accumulation in streptozotocin-induced diabetic rat tissues, J. Pharm. Pharmacol. 53(5):653-668 (2001)) or isolated from plants (De la Fuente, 2003; Benvenuti et al., Identification, characterization, and biological activity of chalcone derivatives of Glycyrrhiza glabra L. Rivista Italiana 7(20):13-16 (1996); Kawanishi et al., Aldose reductase inhibitors from the nature, Curr. Med. Chem. 10:1353-1374 (2003)). Due to toxicity, potency and efficacy problems encountered in preclinical and clinical trials, however, no ALR2 inhibitors have advanced through the process of clinical development.

It is desirable to provide purified compounds from a plant extract having specific activities to act synergistically or independently to provide an anti-diabetic effect. It is also desirable to provide compounds within the extract to effect insulin resistance and modulate enzymes involved in glucose metabolism to facilitate the control of diabetic and non-diabetic symptoms involved in a variety of disorders and diseases found in mammals.

### SUMMARY OF THE INVENTION

This invention comprises an extract of Artemisia dracunculus that can be used for the treatment and prevention of diabetes, metabolic syndrome and other comorbidities that share the underlying commonality of insulin resistance. The invention includes the identity of six compounds from the extract that contribute to the activity of the extract by inhibiting protein tyrosine phosphatase-1B (PTP-1B) activity, phosphoenolpyruvate carboxykinase (PEPCK) gene expression or aldose reductase activity (ALR2). The compounds include 4,5-Di-O-caffeoylquinic acid, davidigenin, 6-demethoxycapillarisin, 2′,4′-dihydroxy-4-methoxydihydrochalcone, 2′,4-dihydroxy-4′-methoxydihydrochalcone and sakuranetin.

Each of the specific activities of the compounds have a common function of countering metabolic changes associated with insulin resistance. This effect is further demonstrated by the universal nature of one of the compounds of 2′,4′-dihydroxy-4-methoxydihydrochalcone that is active for each of the assays addressing different aspects of insulin resistance. This universal activity is not predicted for different enzymes with unique structures or for the expression of genes that are independently regulated. In addition, the compound 6-demethoxycapillarisin is characterized by the ability to inhibit ALR2 activity and PEPCK gene expression. The activity of the Artemisia dracunculus extract in each of the specified assays appears to be dependent upon more than the additive effects of the individual inhibitors. Thus, the use of more than one of the compounds provides unique combinations and interactions of the compounds. Any one of the compounds or any combinations of the compounds may be effective for treating or preventing any condition related to diabetes or metabolic syndrome.

This invention relates to isolated and purified compounds with specific activities within the extract that act synergistically or independently to provide an anti-diabetic effect. Diabetes is a complex disease involving many interconnected metabolic pathways thereby dictating the involvement of multiple pharmacological targets as effective treatment and prevention strategies. The extract of the present invention inhibits PTP-1B activity and PTP-1B gene expression. Compounds of the present invention that inhibit PTP-1B activity and PTP-1B gene expression include 2′,4-dihydroxy-4′-methoxydihydrochalcone, 2′,4′-dihydroxy-4-methoxydihydrochalcone and sakuranetin.

The present invention investigates the ALR2 inhibitory activity of the extract to evaluate its potential for the treatment of diabetic complications that are caused by the enhanced activation of the polyol pathway during hyperglycemia and insulin resistance. Compounds from an extract of the present invention of 4,5-Di-O-caffeoylquinic acid, davidigenin, 6-demethoxycapillarisin, and 2′,4′-dihydroxy-4-methoxydihydrochalcone were identified to inhibit the activity of ALR2.

Compounds from an extract of the present invention of 6-demethoxycapillarisin, and 2′,4′-dihydroxy-4-methoxydihydrochalcone were identified as responsible for decreasing PEPCK expression.

Also encompassed by the invention are pharmaceutical compositions comprising an effective amount of one or more compounds selected from the group consisting of 4,5-Di-O-caffeoylquinic acid, davidigenin, 6-demethoxycapillarisin, 2′,4-dihydroxy-4′-methoxydihydrochalcone, 2′,4′-dihydroxy-4-methoxydihydrochalcone and sakuranetin.

Also encompassed by the invention is the use of a pharmaceutical composition, wherein the use is selected from the group consisting of modulating glucose level in a mammal, modulating insulin resistance in a mammal, modulating insulin-stimulated glucose uptake in a mammal, modulating hepatic glucose level in a mammal, modulating the expression of PEPCK in a mammal, inhibiting PTP-1B activity and PTP-1B gene expression in a mammal, inhibiting ALR2 activity in a mammal, treating type 2 diabetes and hyperglycemia in a mammal.

The invention will be more fully described by reference to the following drawings.

### BRIEF DESCRIPTION OF THE DRAWINGS

FIG. 1A presents a histogram showing inhibition of aldose reductase activity (ALR2) enzyme activity of an extract of Artemisia dracunculus and quercitrin liquid chromatography fractions (HPLC).

FIG. 1B shows inhibition of ALR2 enzyme activity of liquid chromatography fractions (HPLC) of an extract of Artemisia dracunculus and quercitrin.

FIG. 1C shows inhibition of ALR2 enzyme activity of the HPLC fractions of an active subfraction shown in FIG. 1B and quercitrin.

FIG. 1D shows inhibition of ALR2 enzyme activity of the HPLC fractions of an active subfraction shown in FIG. 1B and quercitrin.

FIG. 1E shows inhibition of ALR2 enzyme activity of the HPLC fractions of compounds of an active fraction of FIG. 1D determined by liquid chromatography-mass spectrometry (LCMS) and quercitrin.

FIG. 1F shows inhibition of ALR2 enzyme activity of the HPLC fractions of compounds of an active fraction of FIG. 1D determined by liquid chromatography-mass spectrometry (LCMS) and quercitrin.

FIG. 2 shows effect of phosphoenolpyruvate carboxykinase (PEPCK) expression of HPLC fractions of the Artemisia dracunculus extract. The fractions were tested at 50 μg/ml of media in H4IIE cells.

FIG. 3 presents a histogram showing dose-response effect of 5-demethoxycappilarisin (6-DMX) treatment on PEPCK gene expression in H4IIE cells.

FIG. 4 presents a histogram showing identification of 2′,4′-dihydroxy-4-methoxydihydrochalcone (DMDC) as a compound decreasing PEPCK gene expression level more than 50%.

FIG. 5A shows protein tyrosine phosphatase-1B (PTP-1B) activity of an extract Artemisia dracunculus HPLC fractions.

FIG. 5B shows PTP-1B activity of HPLC fractions of an active fraction shown in FIG. 5A.

FIG. 6 shows the effect of HPLC fractions and subfractions of an extract Artemisia on gene expression of PTP-1B using RT-PCR, each tested at 20 μg/ml of media.

### DETAILED DESCRIPTION

Reference will now be made in greater detail to a preferred embodiment of the invention, an example of which is illustrated in the accompanying drawings. Wherever possible, the same reference numerals will be used throughout the drawings and the description to refer to the same or like parts.

In one embodiment, the presenting invention relates to a method of treating diabetes in a mammal, including humans, specifically type 2 diabetes, by administering an effective amount to a mammal of an extract from a plant such as Artemisia containing one or more compounds selected from 4,5-Di-O-caffeoylquinic acid, davidigenin, 6-demethoxycapillarisin, 2′,4-dihydroxy-4′-methoxydihydrochalcone, 2′,4′-dihydroxy-4-methoxydihydrochalcone and sakuranetin or by administering the one or more compounds per se. Particularly, the plant can be Artemisia dracunculus. In one embodiment, the method can be used for treating hyperglycemia and insulin resistance.

The term “extract” as used herein means a substance or composition obtained from a plant or plant part source, regardless of whether the substance or composition is found external to the plant (i.e., an exudate), is found within the plant or plant part but external to the cells thereof, or is found within the cells of the plant. Chemical and/or physical action, as would be understood in the art, may be required to obtain the substance or composition from the plant or plant part.

The effective amount of the extract may be a dosage that ranges from about 10 mg/kg to about 10,000 mg/kg. For example, the effective dose is 10,000 mg/kg. The exact value of an effective dose varies based upon the sensitivity and size of each patient, and is readily determinable by one of skill in the art using conventional procedures for the routine administration of effective dose. 4,5-Di-O-caffeoylquinic acid (compound 1) is represented by the following structure:

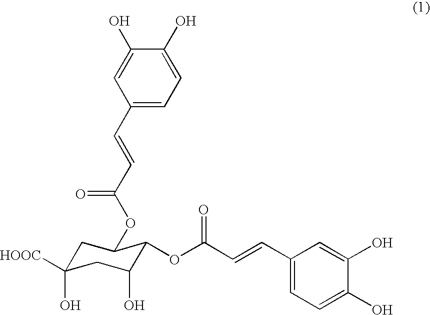

Davidigenin (compound 2) is represented by the following structure:

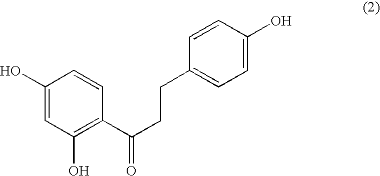

6-demethoxycapillarisin (compound 3) is represented by the following structure:

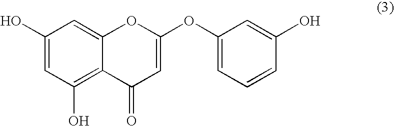

2′,4′-dihydroxy-4-methoxydihydrochalcone (compound 4) is represented by the following structure:

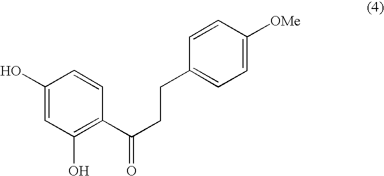

2′,4-dihydroxy-4′-methoxydihydrochalcone (compound 5) is represented by the following structure:

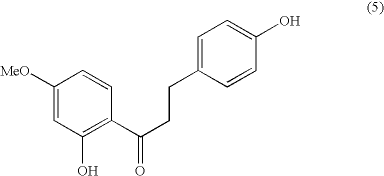

Sakuranetin (compound 6) is represented by the following structure:

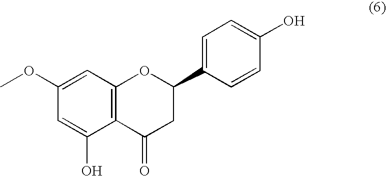

In another embodiment, the present invention relates to a method of modulating protein tyrosine phosphatose-1B (PTP-1B) activity in a mammal comprising administering an effective amount of an extract of Artemisia plant species, in particular, Artemisia dracunculus . The term “modulating” as used herein means changing, adjusting, or varying a property of an organism, tissue, cell, or molecule, including varying the quantity, activity, or capacity of a substance such as glucose or a biomolecule such as a polypeptide. The method decreases PTP-1B gene expression. In one embodiment, the method concerns modulating protein tyrosine phosphatose-1B (PTP-1B) activity in a mammal comprising administering an effective amount of an extract of Artemisia plant species containing one or more compounds selected from 2′,4-dihydroxy -4′-methoxydihydrochalcone, 2′,4′-dihydroxy-4-methoxydihydrochalcone and sakuranetin.

In another embodiment, the present invention relates to a method of modulating hepatic glucose output in a mammal comprising administering an effective amount of an extract from an Artemisia plant species containing one or more compounds selected from 6-demethoxycapillarisin, and 2′,4′-dihydroxy-4-methoxydihydrochalcone. In one embodiment, the present invention relates to a method of decreasing phosphoenol pyruvate carboxykinase (PEPCK) expression. In one aspect, the present invention relates to a method of administering an effective amount of an extract from a plant Artemisia containing 2′,4′-dihydroxy-4-methoxydihydrochalcone or the compound per se for decreasing PEPCK gene expression by more than about 50%.

In another embodiment, the present invention relates to a method of modulating enzyme aldose reductase (ALR2) activity in a mammal by administering an effective amount of an extract from an Artemisia plant species, in particular Artemisia dracunculus. In one embodiment, the method of modulating enzyme aldose reductase (ALR2) activity in a mammal comprises administering an effective amount of an extract from an Artemisia plant species containing one or more compounds selected from 4,5-Di-O-caffeoylquinic acid, davidigenin, 6-demethoxycapillarisin, and 2′,4′-dihydroxy-4-methoxydihydrochalcone.

The Artemisia extracts disclosed herein are extracted using a mildly polar fluid such as an alcoholic solution that does not require further fractionation, e.g., to eliminate or reduce the amount of a mutagen or toxin a method of preparing a mildly polar extract of a plant, such as Artemisia, comprising the steps of: contacting a plant such as Artemisia dracunculus with an elicitor and extracting the Artemisia with a mildly polar fluid (e.g., an alcoholic solution), as described in U.S. Pat. No. 6,893,627 and U.S. Patent Application Publication No. 2005/0069598 A1, each hereby incorporated by reference into this application. As noted above, a preferred plant in the genus Artemisia is Artemisia dracunculus. Elicitors contemplated for the contacting step include those generally known in the art. Elicitors used in the contacting step include chitosan, Trichoderma species (preferably Trichoderma harzianum), acetic acid, methyl salicylate, methyl jasmonate, and PlantShield (Bioworks, Inc., Geneva, N.Y.). Suitable elicitors include 0.8 mM methyl salicylate, 0.1 mM methyl jasmonate, and PlantShield (5 ounces to 12 ounces per 100 gallons). Preferably, the elicitor is 0.1% chitosan or Trichoderna harzianum. A variety of alcohols can be used to extract efficacious materials from Artemisia, including methanol, ethanol, and isopropanol. A preferable alcohol used to extract efficacious materials from Artemisia is ethanol. In yet another preferred method of preparing an alcoholic extract of Artemisia dracunculus , the alcoholic solution comprises at least about 60% ethanol. A preferred method of preparing the alcoholic extract further comprises disrupting the Artemisia dracunculus. The disrupting step can be performed by any method known in the art that results in a loss of the integrity of the plant cell wall and membrane, e.g., by grinding Artemisia using a mortar and pestle or a milling device. Another method of preparing the alcoholic extract further comprises drying the extract at an elevated temperature to reduce methyl eugenol concentration. The extract can be filtered and evaporated. The extract can be freeze dried. The freeze dried extract can be homogenized. Above described compounds 1-6 can be isolated from the homogenized dried extract using chromatography.

For the methods of treatment of this invention, a typical treatment course may comprise administration of multiple doses on a daily basis of a composition comprising one or more compounds of the present invention in an amount effective to treat a disorder such as treating or ameliorating symptoms of diabetes, hyperglycemia, insulin resistance, modulating blood glucose levels, modulating hepatic glucose levels in a mammal, modulating PTP-1B activity, decreasing PEPCK, PEPCK expression, and modulating ALR2 activity in an individual. Such a treatment course may be continued for significant periods of time, for example, three doses per day over three months or even indefinitely. In one embodiment, a presently preferred dosing schedule is one dose per day. The treatment may be continued on an as-needed basis.

The foregoing are only exemplary treatment schedules, and other schedules are contemplated. In each case, the suitability of such schedules and the aforementioned modes of administration are determined by those of skill in the art, using routine procedures. For example, those of skill in the art will be able to take the information disclosed in this specification and optimize treatment regimes for human subjects based on clinical trials performed in accordance with the specification.

Various modes of administration are contemplated for use in delivering the composition containing the extract or compounds of the extract. These include all modes known in the art for delivering therapeutic compositions to a mammal such as a human patient. Modes of administration include e.g., oral, nasal, parenteral (e.g., intravenous, intramuscular and subcutaneous), transdermal and topical. The extract including compounds of the present invention or the compounds per se can be added to a pharmaceutically acceptable formulation, nutraceutical, and/or functional food in any suitable amount. In one embodiment, the pharmaceutically acceptable formulation, nutraceutical, and/or functional food comprises the compound in an amount of at least 0.1% by weight to about 95% by weight.

Pharmaceutical compositions comprising a mildly polar extract of Artemisia including one or more compounds of 4,5-Di-O-caffeoylquinic acid, davidigenin, 6-demethoxycapillarisin, 2′,4-dihydroxy-4′-methoxydihydrochalcone, 2′,4′-dihydroxy-4-methoxydihydrochalcone and sakuranetin, or the compounds per se, and one or more pharmaceutically acceptable formulation agents are also encompassed by the invention. The pharmaceutical compositions are used to provide therapeutically effective amounts of the compounds from the extract of Artemisia (e.g., Artemisia dracunculus ) of the present invention. The invention also provides for devices to administer the extract encapsulated in a membrane.

In one embodiment, the pharmaceutical compositions containing the extracts or one or more compounds of the extract of Artemisia or the compounds per se may be in any form suitable for oral use, such as e.g., tablets, troches, lozenges, aqueous or oily suspensions, dispersible powders or granules, emulsions, hard or soft capsules, or syrups or elixirs. Compositions intended for oral use can be prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations.

According to the invention, tablets contain the active ingredient(s) in admixture with non-toxic pharmaceutically acceptable excipients, such as inert diluents, granulating, disintegrating and lubricating agents, which are suitable for the manufacture of tablets. Binders may be used to hold the composition comprising the extract or its constituents together to form a hard tablet. Exemplary binders include materials from natural products such as acacia, tragacanth, starch and gelatin. Other suitable binders include methyl cellulose (MC), ethyl cellulose (EC), and carboxymethyl cellulose (CMC). The tablets may be uncoated or they may be coated by known techniques to delay disintegration and absorption in the gastrointestinal tract and thereby provide a sustained action over a longer period. The formulations can also be so constituted that they release the active ingredient only or preferably in a particular part of the intestinal tract, possibly over a period of time. Such formulations would involve coatings, envelopes, or protective matrices which may be made from polymeric substances or waxes.

Formulations for oral use may also be presented as hard gelatin capsules wherein the active ingredient is mixed with an inert solid diluent, or as soft gelatin capsules wherein the active ingredients is mixed with water or an oil medium.

Aqueous suspensions contain the active material in admixture with excipients suitable for the manufacture of aqueous suspensions, such as e.g., suspending agents, dispersing or wetting agents, preservatives, coloring agents, flavoring agents, and sweetening agents. Dispersible powders and granules suitable for preparation of an aqueous suspension by the addition of water provide the active ingredient(s) in admixture with a dispersing or wetting agent, suspending agent and one or more preservatives. Additional excipients, for example sweetening, flavoring and coloring agents, may also be present.

The compositions of the present invention also may be formulated as a food or beverage additive as defined by the U.S. Food and Drug Administration. In one embodiment, the compositions of the present invention include at least one formulation agent selected from the group consisting of diluents, fillers, salts, binders and biologically acceptable carriers.

Pharmaceutically acceptable carrier preparations for parenteral administration include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. The active therapeutic ingredient may be mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

It will be appreciated that the treatment methods of the invention are useful in the fields of human medicine and veterinary medicine. Thus, the subject or individual to be treated may be a mammal, preferably human, or other animals. For veterinary purposes, subjects include, for example, farm animals such as cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

As noted above, Artemisia extracts may be combined with a variety of substances in methods to treat, or ameliorate the symptoms of, diabetes. For example, an effective dose of an Artemisia extract may be combined with an effective dose of any one of the following naturally occurring (e.g., plant-based) substances or chemical compounds: gymnema sylvestre, fenugreek, bitter melon, alpha-lipoic acid, banaba Leaf, yacou root, momordica charantia, olive leaf extract, pterocarpus marsupium, salacia reticulate, garlic, hawthorn, corosolic acid, ursolic acid, D-pinitol, aloe vera, chromium picolinate, phosphatidylserine, omega 3 fatty acids, resistant starch, catharanthus roseus, anacardium occidentale, syzygium cumini, eucalyptus globules, lupinus albus, allium cepa, allium sativum, tecoma stans, urtica dioica, taraxacum officinale, kyllinga monocephala, phyllanthus emblica, phyllanthus niruri, azadirachta indica, morbus alba, poterium ancistroides, and daucus carota. These combinations of substances for use in methods for treating, or ameliorating the symptoms of, diabetes provide the benefits attributable to each component (i.e., the Artemisia extract and the substance with which it is combined for administration).

Also as noted above, Artemisia extracts may be combined with a variety of substances in methods of improving nutrition, such as sports nutrition. In such methods, an effective dose of an Artemisia extract is combined with an effective dose of any one of the following naturally occurring (e.g., plant-based) substances or chemical compounds: creatine, creatine monohydrate, creatine salts such as creatine citrate, creatine pyruvate, creatine derivatives and salts thereof, phosphocreatine, caffeine, alpha-lipoic acid, glucosamine, chondroitin, hydrolyzed collagen, methylsulfonyl-methane, whey protein, L-glutamine, phosphatidylcholine, choline, choline salts, phosphatidylserine, beta-hydroxy beta-methylbutyrate, pyruvate, L-carnitine, D-ribose, an amino acid (a conventional amino acid), a branched chain amino acid, S-adenosylmethionine, taurine, conjugated linoleic acid, alpha-lipoic acid, alpha-lipoic acid salts, and glycerin. In referencing the salts of various compounds, the invention contemplates the compound and any suitable salt-forming counterions (such as alkali metal ions, alkaline earth metal ions, halogen ions, organic cations, organic ions, complex ions and any other counterion known in the art (preferably sodium)). These combinations of substances for use in methods for improving nutrition, such as sports nutrition, also provide the benefits attributable to each component (i.e., the Artemisia extract and the substance with which it is combined for administration).

Further, as noted above, Artemisia extracts may be combined with a variety of substances in methods for weight control. In such methods, an effective dose of an Artemisia extract is combined with an effective dose of any one of the following naturally occurring (e.g., plant-based) substances or chemical compounds: pyruvate, L-carnitine, hydroxycitric acid, ephedrine, caffeine, and conjugated linoleic acid (CLA). These combinations of substances for use in methods for improving nutrition, such as sports nutrition, also provide the benefits attributable to each component (i.e., the Artemisia extract and the substance with which it is combined for administration). Although the same combination of substances may be useful in more than one method, the methods are nonetheless distinguishable based on purpose.

The following examples are provided to describe the invention in greater detail, and are intended to illustrate, not to limit, the appended claims. Example 1 describes the preparation of an extract of Artemisia dracunculus . Example 2 describes purification, isolation and identification of compounds from the extract of the Artemisia dracunculus extract. Example 3 described liquid chromatography-mass spectrometry analysis. Example 4 describes an assay for ALR2 enzyme. Example 5 describes assays for PTP-1B Activity and PTP-1B gene expression. Example 6 describes assays for PEPCK activity and gene expression. Example 7 describes isolation of pure compounds with ALR2 inhibitory activity. Example 8 describes isolation of pure compounds with PEPCK gene expression inhibitory activity. Example 9 describes isolation of pure compounds with PTP-1B inhibitory activity. Example 10 describes identified compounds contributing to the anti-diabetic activity of the extract of Artemisia . Example 11 describes pharmaceutical compositions and administration.

### EXAMPLE 1

### Extract Preparation

The seeds of Artemisia dracunculus L. were purchased from Sheffield's Seed Co., Inc. (Locke, N.Y.). The plants were grown in hydroponics and harvested as the total plant material above the root mass. The harvested plants were frozen and stored at −20° C. prior to extraction. Four kilograms of the shoot material was heated to 80° C., with 12 liters of 80% ethanol (v/v) for 2 hours. The extraction was continued for an additional 10 hours at 20° C. The extract was then filtered through cheesecloth and evaporated with a rotary evaporator and the final volume was reduced to 1 liter. The aqueous extract was freeze dried for 48 hours and the dried extract was homogenized with a motor and pestle.

### EXAMPLE 2

### Purification, Isolation and Identification of Compounds from the Extract

Purification and isolation of compounds were carried out using a preparatory HPLC from Waters consisting of W717 plus auto sampler, W600E multi solvent delivery system, W600 controller, W490E multi wavelength detector and Waters fraction collector. LC/MS system used for analysis includes the Waters (Milford, Mass.) LC-MS Integrity™ system consisting of a solvent delivery system with a W616 pump and W600S controller, W717plus auto-sampler, W996 PDA detector and Waters TMD Thermabeam™ electron impact (EI) single quadrupole mass detector with fixed ionization energy of 70 eV. Data were collected and analyzed with the Waters Millennium® v. 3.2 software, linked with the 6^{th} Edition of the Wiley Registry of Mass Spectral Data, containing 229,119 EI spectra of 200,500 compounds. After the 996 PDA detector the eluent flow was split into two equal flow paths with an adjustable flow splitter, model 600-PO10-06 (Analytical Scientific Instruments, El Sobrante, Calif.). One of them was to the Thermabeam EI mass detector, and the other to a Varian 1200L (Varian Inc., Palo Alto, Calif.) triple quadrupole mass detector with electrospray ionization interface (ESI), operated in either positive, or negative ionization mode. The electrospray voltage was −4.5 kV, heated capillary temperature was 240° C., sheath gas air for the negative mode, and electrospray voltage 5 kV and sheath gas nitrogen for the positive ionization mode; mass detector scanning from 110 to 1400 atomic mass units. Data from the Varian 1200L mass detector was collected and compiled using Varian's MS Workstation, v. 6.41, SP2. The ¹H, ¹³C- NMR spectra and 2D-NMR experiments were recorded using a Bruker Avance AV-300 NMR spectrometer at 300 MHz (¹H) and 75 MHz (¹³C). The 2D experiments ¹H-¹H COSY (Correlation Spectroscopy), HMBC, and edited-HSQC (Heteronuclear Single Quantum Coherence) were acquired using standard Bruker software. All compounds were measured in CD₃OD. For the enzyme assay, a Beckman spectrophotometer (DU Series 600), which operates in the wavelength range of 190 to 1100 nm was used to measure the change in absorbance of NADPH.

### High Performance Liquid Chromatography Analysis

One gram of the dried extract was dissolved in 5 ml of 60% ethanol and 0.5 ml of acetonitrile and purified using a preparatory HPLC. For the initial purification, a Waters 19×300 mm symmetry prep, C8, reverse phase column with a particle size of 7 μm was used. The mobile phases consisted of two components: Solvent A (0.5% ACS grade acetic acid in double distilled de-ionized water, pH 3-3.5), and Solvent B (100% Acetonitrile). For the initial separation, a gradient run of 5% B to 95% B over 35 minutes was used at a flow rate of 8 ml/m. Ten fractions at five minutes interval were collected and tested with each of the assay systems measuring anti-diabetic activity. Fractions and sub-fractions that showed higher percent inhibition of the enzyme were further purified using different conditions (Table 1). The ultra violet (UV) profiles were monitored at wavelengths of 210 and 290 nm.

TABLE 1
Conditions used at different steps of purification
of the ethanolic extract of Artemisia dracunculus.

| Fractions | Column type | Mobile phase | Type of elution | Flow rate |
|---|---|---|---|---|
| F 7 | Waters | 100% acetonitrile | Gradient elution: 30%-95% | 8 ml/m |
| | RP C8, 7 μm | 0.5% acetic acid | acetonitrile over 120 minutes | |
| | 19 × 300 mm | in water | | |
| F 7-2 | Waters | 100% acetonitrile | Isocratic elution: | 1 ml/m |
| | RP C8, 7 μm | 100% methanol | Acetonitrile 20% | |
| | 7.5 × 300 mm | 0.5% acetic acid | Methanol 30% | |
| | | in water | 0.5% acetic acid in water 50% | |
| F 7-9 | Waters | 100% acetonitrile | Gradient elution: 30%-95% | 1 ml/m |
| | RP C8, 7 μm | 0.5% acetic acid | Acetonitrile over 90 minutes | |
| | 7.5 × 300 mm | in water | | |
| F 5 | Waters | 100% acetonitrile | Gradient elution: 20%-95% | 5 ml/m |
| | RP C8, 7 μm | 0.5% acetic acid | Acetonitrile over 100 minutes | |
| | 19 × 300 mm | in water | | |
| F 5-7 | Phenomenex | 100% acetonitrile | Gradient elution: 20%-95% | 5 ml/m |
| | RPC18, 5 μm | 0.5% acetic acid | Acetonitrile over 70 minutes | |
| | 10 × 250 mm | in water | | |

### EXAMPLE 3

### Liquid Chromatography-Mass Spectrometry Analysis

Substances were separated on a Phenomenex® Luna C-8 reverse phase column, size 250×4.6 mm, particle size 5 μm, equipped with a Phenomenex® SecurityGuard™ pre-column. The mobile phase consisted of two components: Solvent A (0.5% ACS grade acetic acid in double distilled de-ionized water, pH 3-3.5), and Solvent B (100% Acetonitrile). The mobile phase flow was adjusted at 0.5 ml/m, and a gradient of 15% B to 95% B over 30 minutes was used.

Physical Properties of Compound 1 (4,5-Di-O-caffeoylquinic acid)

UV λₘₐₓ (acetonitrile): 218, 243, 327. EI MS m/z (% rel. int.): 182 (19), 163 (15), 149 (12), 136 (51), 123 (100), 110 (75), 94 (44); (−) ESI MS m/z (% rel. int.): 515 (100) [M−H]⁻, 1031 (4) [2×M−H]. ¹H NMR (CD₃OD, 300 MH_{z}) 67 7.60 and 7.52 (1H each, d, J=15.9 H_{z}, H-7′, 7″), 7.02 and 7.00 (1H each, d, J=2.0 H_{z}, H-2′, 2″), 6.92 and 6.90 (1H each, dd, J=8.1, 2.1 H_{z}, H-6″, 6″), 6.75 and 6.74 (1H each, d, J=8.1 H_{z}, H-5′,5″), 6.28 and 6.19 (1H each, d, J=15.9 H_{z}, H-8′, 8″), 5.64 (1H, brs, H-5), 5.12 (1H, dd, J=8.8, 2.6 H_{z} H-4), 4.36 (1H, brs, H-3), 1.92-2.34 (4H, m, H-2, 6); ¹³C NMR (CD₃OD, 300 MH_{z,}) 67 175.7 (C-7), 167.2 and 166.8 (C-9′, 9″), 148.3 (C-4′, 4″), 146.3 and 146.2 (C-7′, 7″), 145.4 (C-3′, 3″), 126.3 and 126.2 (C-1′1″), 121.7 (C-6′, 6″), 115.1 (C-5′, 5″), 113.8 (C-2′, 2″), 113.4 and 113.3 (C-8′, 8″), 74.8 (C-1), 74.5 (C-4), 68.1 (C-3), 67.7 (C-5), 38.1 (C-6), 37.0 (C-2). The compound was described as 4,5-Di-O-caffeoylquinic acid, as described in Zhu et al., Phenolic Compounds from the leaf extract of Artichoke (Cynara scolymus L.) and their antimicrobial activities, Journal of Agricultural and Food Chemistry 52(24):7272-7278 (2004).

Physical Properties of Compound 2 (Davidigenin)

UV λₘₐₓ (acetonitrile): 216, 277, 312. EI MS m/z (% rel. int.): 258 (80), 239 (28), 223 (3), 152 (8), 137 (100), 120 (24), 107 (40), 91 (4), 77 (10); (−)ESI MS m/z (% rel. int.): 257 (100) [M−H]⁻, 151 (6). 1H NMR (acetone-d₆, 300 MH_{z )} 67 7.85 (1H, d, J=8.7 H_{z}, H-6′), 7.13 (2H, d, J=8.4 H_{z}, H-2 and 6), 6.77 (2H, d, J=8.4 H_{z}, H-3 and 5), 6.44 (1H, dd, J=8.7, 1.8 H_{z}, H-5′), 6.34 (1H, d, J=1.8 H_{z}, H-3′), 3.26 (2H, t, J=7.7 H_{z}, H-α), 2.93 (2H, t, J=7.7 H_{z}, H-β); ¹³C NMR (acetone-d₆, 75 MH_{z )} 67 204.2 (C═O), 165.0 (C-2′), 164.4 (C-4′), 155.6 (C-4), 132.8 (C-6′), 131.9 (C-1), 129.3 (C-2, 6), 115.0 (C-3, 5), 113.0 (C-1′), 107.8 (C-5′), 102.5 (C-3′), 39.6 (C-α), 29.3 (C-β). The compound was identified as Davidigenin as described in Jensen et al., Dihydrochalcones from Viburnum davidii and V. lantanoides. Phytochemistry 16:2036-2038 (1977).

Physical Properties of Compound 3 (6-demethoxycapillarisin)

UV λₘₐₓ (acetonitrile): 197, 230, 285. EI MS m/z (% rel. int.): 286 (100), 269 (2), 257 (8), 229 (3), 194 (4), 153 (35), 134 (19), 121 (5), 106 (8); (+)ESI MS m/z (% rel. int.): 287 (100) [M+H]^{+,} 245 (8), 195 (11) ¹H NMR (CD₃OD, 300 MH_{z}) 67 7.09 (2H, d, J =9.0 H_{z}, H-2′, 6′), 6.88 (2H, d, J=9.0 H_{z}, H-3′, 5′), 6.31 (1H, d, J=2.1 H_{z}, H-8), 6.20 (1H, d, J=2.1 H_{z}, H-6), 5.11 (1H, s, H-3); ¹³C NMR (CD₃OD, 300 MH_{z}) δ 185.6 (C-4), 170.3 (C-2), 165.9 (C-7), 163.3 (C-5), 157.8 (C-4′), 157.2 (C-9), 145.3 (C-1′), 123.0 (C-2′, 6′), 117.7 (C-3′, 5′), 103.6 (C-10), 100.7 (C-6), 95.2 (C-8), 88.0 (C-3). The compound was described as 6-demethoxycapillarisin in Sharon et al., Isolation, purification, and identification of 2-(p-hydroxyphenoxy)-5,7-dihydroxychromone: a fungal-induced phytoalexin from Cassia obtusifolia. Plant physiology. 98:303-308 (1992).

Physical Properties of Compound 4 (2′,4′-dihydroxy-4-methoxydihydrochalcone)

UV λₘₐₓ (acetonitrile): 215, 276, 312. EI MS m/z (% rel. int.): 272 (46), 253 (15), 166 (9), 151 (100), 137 (4), 120 (23), 107 (20), 95 (7); (−)ESI MS m/z (% rel. int.): 165 (100), 271 (89) [M−H]⁻, 541 (22) [2×M−H]⁻. ¹H NMR (acetone-d₆, 300 MH_{z}) 67 7.84 (1H, d, J=8.7 H_{z}, H-6′), 7.23 (2H, d, J=8.4 H_{z}, H-2 and 6), 6.85 (2H, d, J=8.4 H_{z}, H-3 and 5), 6.43 (1H, dd, J=8.7, 1.8 H_{z} , H-5′), 6.33 (1H, d, J=1.8 H_{z}, H-3′), 3.77 (3H, s, OCH₃), 3.28 (2H, t, J=7.7 H_{z} , H-α), 2.96 (2H, t, J=7.7 H_{z}, H-β); ¹³C NMR (acetone-d₆, 75 MH_{z}) 67 204.0 (C═O), 164.8 (C-2′), 164.7 (C-4′), 158.2 (C-4), 133.1 (C-1′), 132.8 (C-6), 129.3 (C-2, 6), 113.7 (C-3, 5), 112.9 (C-1′), 107.9 (C-5′), 102.6 (C-3′), 54.5 (OCH₃), 39.4 (C-α), 29.7 (C-β). Compound 4 was described as 2′,4′-dihydroxy-4-methoxydihydrochalcone which has very similar NMR data to davidigenin as described in Jensen et al., Dihydrochalcones from Viburnum davidii and V. lantanoides. Phytochemistry 16:2036-2038 1977 except it also has an O-methyl group.

Additional NMR data for Compound 4 is shown in the following table 2

TABLE 2

| C/H | δ_{C} | δ_{H}(int., mult., J in H_{Z}) | HMBC (¹³C No.) |
|---|---|---|---|
| 1 | 133.1 | | |
| 2 | 129.3 | 7.23 (1H, d, 8.4) | 3, 4, 6, β |
| 3 | 113.7 | 6.85 (1H, d, 8.4) | 1, 4, 5 |
| 4 | 158.2 | | |
| 5 | 113.7 | 6.85 (1H, d, 8.4) | 1, 3, 4 |
| 6 | 129.3 | 7.23 (1H, d, 8.4) | 2, 4, 5, β |
| 1′ | 112.9 | | |
| 2′ | 164.8 | | |
| 3′ | 102.6 | 6.33 (1H, d, 1.8) | 1′, 2′, 4′, 5′ |
| 4′ | 164.7 | | |
| 5′ | 107.9 | 6.43 (1H, dd, 8.7, 1.8) | 1′, 3′ |
| 6′ | 132.8 | 7.84 (1H, d, 8.7) | 2′, 4′, C═O |
| C═O | 204.0 | | |
| α | 39.4 | 3.28 (2H, t, 7.7) | 1, C═O, β |
| β | 29.7 | 2.96 (2H, t, 7.7) | 1, 2, 6, C═O, α |
| OCH₃ | 54.5 | 3.77 (3H, 5), | 4 |

Physical Properties Compound 5 (2′,4-dihydroxy-4′-methoxydihydrochalcone)

Positive identification with NMR data was performed.

Physical Properties Compound 6 (Sakuranetin)

Sakuranetin was positively identified by LC-MS with mass spectral matching using the Wiley library of mass spectra. Its identity was further confirmed with a commercially available authentic chemical standard of sakuranetin by LC retention time, mass spectra and UV profiles as well as NMR.

### Assays for Activity Guided Fractionation

### EXAMPLE 4

### ALR2 Enzyme Assay

Human recombinant ALR2 enzyme was purchased from Wako Chemicals USA Inc. Enzyme activity was measured at each step of purification of the extract, by monitoring the decrease in NADPH absorbance at a wavelength of 340 nm (Nishimura et al., Purification and characterization of recombinant aldose reducatse expressed in baculovirus system. Biochim. Biophys. acta. pp. 1078-1171 (1991)), using a spectrophotometer. One hundred micro liters of the reaction mixture contained 100 mM sodium phosphate buffer (pH 6.2), 0.15 mM NADPH, 10 mM DL-Glyceraldehyde and 1 mU of human recombinant ALR2 enzyme. The samples were prepared in 10% DMSO and the final concentration of the samples or the positive control quercitrin was 3.75 μg/ml. The reaction was initiated by adding the enzyme and the change in NADPH absorbance was monitored over seven minutes.

### EXAMPLE 5

### PTP-1B Inhibitory Assays

### PTB-1B Activity

The activity of PTP-1B was assayed by hydrolysis of p-nitrophenol phosphate (PNPP). Skeletal muscle cells were incubated overnight (16 hours) with test substance (extract, fractions or pure compounds) at 20 μg/ml of media. Cell lysate was prepared and PTP-1B was immunoprecipitated with specific antibody (Upstate Biotechnology, Lake Placid, N.Y.). The immunoprecipitate was incubated in Phosphatase Reaction Buffer (20 mmol/L HEPES, pH 7.4, 150 mmol/L NaCl, 5 mM dithiothreitol, 1 mmol/L PNPP) for 20 minutes at 37° C. The reaction was stopped with 0.2 mol/L NaOH, and the absorbance at 410 nm was measured. The reactions were run in triplicate (Moeslein et al., The CLK family kinases, CLK1 and CLK2, phosphorylate and activate the tyrosine phosphatase, PTP-1B. J Biol Chem, 274:26697-26704 (1999)).

### Quantitative Real Time PCR for PTP-1B Gene Expression

Cells were incubated overnight (16 hours) with test substance (extract, fractions or pure compounds) at 20 μg/ml of media and harvested from the culture treatments at the designated time points. After extraction and quantification of RNA, quantitative real time PCR (qPCR) analysis was carried out using Taqman® one-step RT-PCR Master Mix (Applied Biosystems, Branchburg, N.J.). 20 ng of total RNA were added per 50 μl reaction with sequence-specific primers (200 nM) and Taqman® probes (200 nM) as indicated here, PTP-1B probe; 5′FAM d(AGTGATGGAGAAAGGTT)BHQ-1 3′. PTP-1B forward primer; 5′ d(GGGTGTCGTCATGCTCAACA)3′ and PTP-1B reverse primer; 5′ d(GCCAGTATTGTGCGCATTTTAA)3′. Primers and probes were designed by and purchased from Applied Biosystems. qPCR assays were carried out in triplicate on an ABI Prism 7700 sequence detection system. Thermocycling conditions were 48° C. for 30 minutes (reverse transcription) and 95°C. for 10 minutes (initial denaturation) followed by 40 cycles at 95° C. for 15 seconds (denaturation) and 60° C. for 45 seconds (annealing and extension). The threshold was set above the non-template control background and within the linear phase of target gene amplification to calculate the cycle number at which the transcript was detected (denoted C_{T}). Gene expression values were calculated based on the standard curve of each target gene (RNA of control cells with two-fold serial dilutions from 200 ng to 3.125 ng) and normalized by the reference housekeeping gene β-Actin (1). Results were expressed as percentage change of control (Applied Biosystems (2001) Applied Biosystems User Bulletin Number 2, Foster City, Calif.).

### EXAMPLE 6

### PEPCK Gene Inhibition Assay

### Cell Culture

H4IIE hepatoma cells (ATCC CRL-1600) were plated in 24-well tissue culture plates (Greiner Bio One) and were grown to confluence in Dulbecco's modified Eagle's medium (DMEM) containing 2.5% (v/v) newborn calf serum and 2.5% (v/v) fetal calf serum. Cells were treated for 8 hours with 500 nM dexamethasone and 0.1 mM cAMP (Dex/cAMP, both Sigma) to induce PEPCK gene expression and different concentrations or volumes of each of the tested compounds, plant extract or 10 nm of Insulin. The fractions were tested at 50 μg/ml of media and the compounds were tested at the doses of 2.5 μg/ml, 5 μg/ml and 10 μg/ml and 25 μg/ml. Three wells were allocated for each treatment as well as for a negative control (untreated cells).

### Cell Viability Assay and Dose Range Determination

Cell viability was measured by the MTT assay (Mosmann, Rapid colorimetric assay for cellular growth and survival: application to proliferation and cytotoxicity assays. Journal of Immunological Methods, 65:55-63 (1983)). The MTT (3-(4,5-dimethylthiazol-2-yl)-2,5-diphenyl-tetrazolium bromide) (Sigma, St. Louis, Mo.) tetrazolium dye assay was performed to measure cell survival after of incubation with treatments in cell culture assays. MTT (100 μg/ml) was added to the medium in each well and plates were incubated in the cell growth chamber for 5 hours. Medium was then removed and dimethyl sulfoxide (150 μL) was added to each well to solubilize the purple formazan crystals created by mitochondrial dehydrogenase reduction of MTT. After 5 minutes of additional incubation, absorbance was read at 550 nm on a micro plate reader spectrophotometer (Molecular Devices, Sunnyvale, Calif.). The concentrations of test reagents that showed significant cell viability compared to that of the control (dimethyl sulfoxide, 0.1%) were further selected for in vitro gene expression assays. All treatments were performed in duplicate.

### Total RNA Extraction, Purification and cDNA Synthesis

Total RNA was extracted from H4IIE rat hepatoma cells using Trizol reagent (Invitrogen Inc.) following the manufacturer instructions. RNA was quantified spectro-photo-metrically by absorption measurements at 260 nm and 280 nm using the NanoDrop system (NanoDrop Technologies Inc. Delware, USA). Quality of RNA was assessed by separation in gel-electrophoresis. RNA was then treated with Dnasel (Invitrogen Inc.) following the manufacturer guidelines, to remove any traces of DNA contamination. The cDNAs were synthesized using 2.5 μg of RNA for each sample using Stratascript Reverse Transcriptase (Stratagene, La Jolla, Calif.), following the manufacturers'protocol.

### Quantitative Polymerase Chain Reaction (qPCR) and Data Analysis

The synthesized cDNAs were diluted 4-fold. 5 μl of each of these diluted samples were used for PCR reactions of 25 μl final volume. The other components of the PCR reactions were 0.5 μl of 6 μM gene specific primers (synthesized by IDT Inc. USA), 12.5 μl of Brilliant SYBR green PCR master mix (2×) (Stratagene, La Jolla, Calif.) containing green jump-start Taq ready mix. ROX (Stratagene, La Jolla, Calif.) was used as an reference dye. The primers were selected using the Primer Express® vers. 2.0 software (Applied Biosystem, Foster City, Calif.) as follows:

β-actin;
forward primer: 5′- GGGAAATCGTGCGTGACATT -3′
reverse primer: 5′- GCGGCAGTGGCCATCTC -3′
PEPCK;
forward primer: 5′- GCAGAGCATAAGGGCAAGGT -3′
reverse primer: 5′- TTGCCGAAGTTGTAGCCAAA -3′.

β-actin primers were selected from the RefSeq sequence with the accession number NM_{—}031144. Both primers reside on exon 4 of the rat β-actin gene (RGSC assembly v3.4). These primers generated a 76-bp product from β-actin mRNA. PEPCK primers were selected from the RefSeq sequence with the accession number NM_{—}198780. The intron-spanning forward primer was selected to cover Exon9-Exon10 boundary. The reverse primer was selected from Exon 10. These primers generated a 74-bp product from PEPCK mRNA and a 207 bp product from genomic DNA.

Real-time PCR amplifications were performed on MX3000p system (Stratagene, La Jolla, Calif.) using 1 cycle at 50° C. for 2 minutes, 1 cycle of 95° C. for 10 minuets in, followed by 40 cycles of 15 seconds at 95° C. and 1 minute at 60° C. The dissociation curve was completed with one cycle of 1 minute at 95° C., 30 seconds of 55° C. and 30 seconds of 95° C. NRT (non-RT control) and NTC (no template control) were included in each experiment as quality control steps.

RNA expressions for PEPCK, normalized with respect to the expression of housekeeping β-actin gene, were analyzed using the ΔΔCt method (Winer et al., Development and validation of real-time quantitative reverse transcriptase-polymerase chain reaction for monitoring gene expression in cardiac myocytes in vitro, Analytical Biochemistry 270:41-9 (1999)). The ΔΔCt values obtained from these analyses directly reflect the relative mRNA quantities for the specific gene in response to a particular treatment as compared to a calibrator. The dexamethasone/cAMP treatment (positive control) served as a calibrator sample in this study. The value of the PECPK gene expression in the calibrator sample was assigned to 1.0. A value less than 1.0 indicates transcriptional down-regulation (inhibition of gene expression) as compared to the calibrator. Amplification of specific transcripts was further confirmed by obtaining melting curve profiles. All samples were run in duplicate.

### Activity Guided Fractionation of Pure Compounds

### EXAMPLE 7

### Isolation of Pure Compounds with ALR Inhibitory Activity

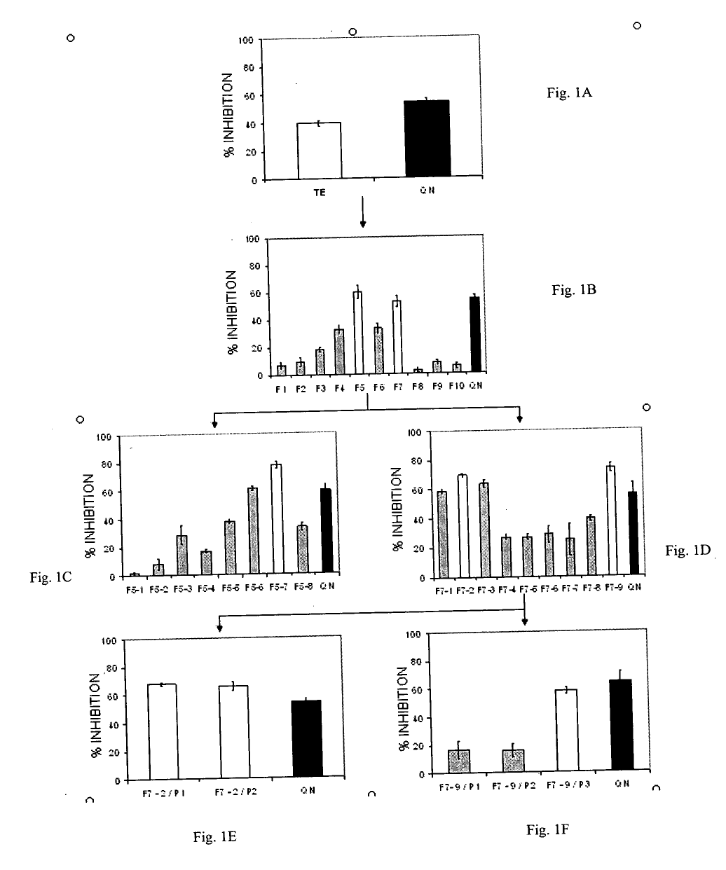

At 3.75 μg/ml, the ethanolic extract of Artemisia dracunculus shoots inhibited the human recombinant ALR2 enzyme activity by 40% while the pure compound, quercitrin, had an inhibition of 54% (FIG. 1A). TE represents Total Extract, QN represents Quercitrin, F represents Fraction, P represents Peak. Quercitrin is a flavonoid that is a well-known inhibitor of the ALR2 enzyme (Matsuda et al., Antidiabetogenic constituents from several natural medicines, represents Pure Appl. Chem. 74(7):1301-1308 (2002); Lee, Cuminaldehyde: Aldose reductase and Glucosidase inhibitor derived from Cuminum cyminum L. seeds, Journal of Agricultural and Food Chemistry 53(7):2446-2450 (2005)) and was used in this study as a positive control. In order to begin the process of identifying the active compounds within the extract, the extract was divided into 10 fractions (based on elution time) by high performance liquid chromatography (HPLC), which were then tested for ALR2 inhibitory activity. Of the ten fractions collected from the total extract, fractions 5 and 7 showed significantly higher inhibition of ALR2 at 3.75μg/ml than the other fractions and a similar inhibition as quercitrin (FIG. 1B). These two fractions were selected for further purification.

Fractionation of the active fraction, F5, yielded eight sub-fractions. The aldose reductase inhibition assay revealed two active sub-fractions, F5-6 and F5-7 (FIG. 1C). Sub-fraction 5-7, after another step of purification gave a single pure compound which inhibited the enzyme by 78% at 3.75 μg/ml, which is 19% greater inhibition than that caused by quercitrin.

Nine sub fractions were collected from the purification of the compounds in F7.

Of these nine sub-fractions F7-1, F7-2, F7-3, and F7-9 showed similar or higher inhibitory activity than quercitrin (FIG. 1D) when tested at 3.75 μg/ml. Since F7-2 and F7-9 showed a slightly higher percent inhibition compared to F7-1 and F7-3, these two sub-fractions were selected for additional purification.

Further purification of the active sub-fraction, F7-2, yielded two different compounds as determined by Liquid Chromatography-Mass Spectrometry (LC-MS) analysis. These two compounds had similar ALR2 inhibitory activity. F7-2/P1 and F7-2/P2 and slightly higher ALR2 inhibitory activity than quercitrin when tested at 3.75 μg/ml (FIG. 1E). Further purification of the other active sub-fraction, F7-9, of F7, revealed that the sub-fraction consisted of at least three different compounds. When the ALR2 inhibition assay was done with these compounds, F7-9/P3 at 3.75 μg/ml showed the highest inhibition at 58.2%, almost similar to quercitrin (FIG. 1F). The purified compounds within the active fractions were identified by a combination of LC-MS and nuclear magnetic resonance (NMR) analysis as described in Example 3. The other fractions or sub-fractions with lower ALR2 inhibitory activity were not further characterized, although they may contain compounds that have high activity, but present in low concentration.

### EXAMPLE 8

### Isolation of Pure Compounds with PEPCK Gene Expression Activity

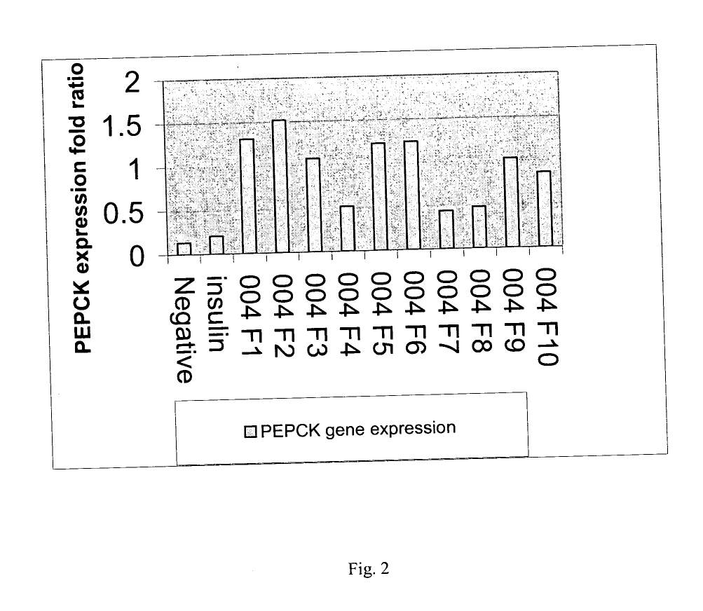

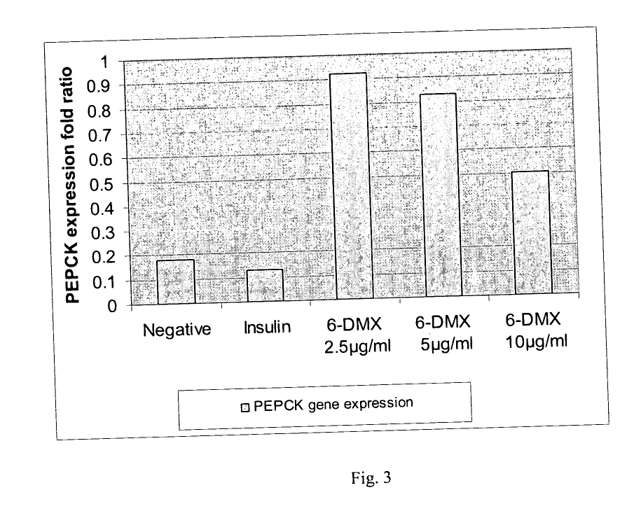

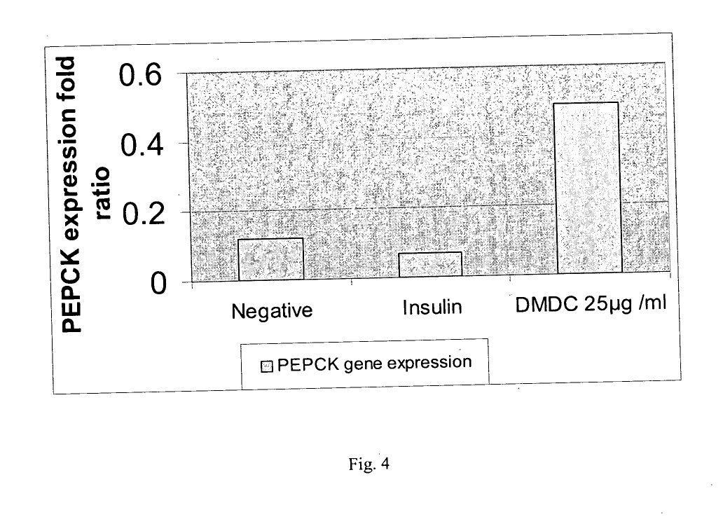

A screen for PEPCK inhibitory activity was developed in hepatic cell cultures using RT-PCR (as described above) to evaluate fractions and isolated pure compounds from an extract of Artemisia dracunculus. Of the 10 fractions tested for activity by treatment of hepatoma cells, fractions 7 was most consistently able to decrease PEPCK expression by greater than 50%, in a manner similar to the positive control of insulin, although not to the same extent (FIG. 2). Fractions 4 and 8 did not consistently promote PEPCK inhibitory activity. Subfractionation of fraction 7 into single compounds by additional HPLC, just as described for the aldose reductase inhibitors, lead to the isolation of compounds 3 and 4. Compounds 3 and 4 were able to repress dexamethazone/cAMP-induced PEPCK gene expression by 68% at 10 μg/ml and more than 50% at 25 μg/ml, respectively (FIGS. 3 and 4). From the dose-response curve presented in FIG. 3, the IC50 for compound 3 (6-demethoxycapilarisin) was calculated to be approximately 25 mM. The other fractions or sub-fractions with lower inhibitory activity over PEPCK expression were not further characterized even though they may contain compounds that have high activity, but present in low concentrations.

### EXAMPLE 9

### Isolation of Pure Compounds with PTP-1B Inhibitory Activity

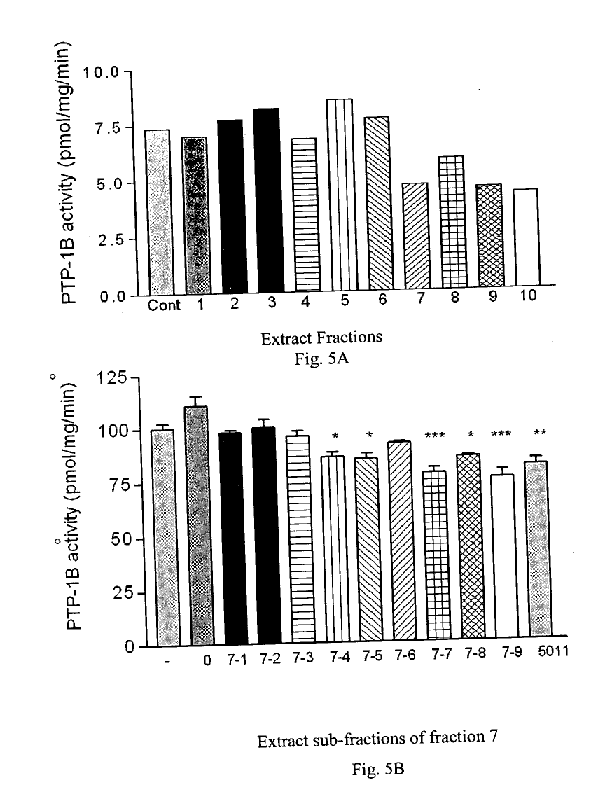

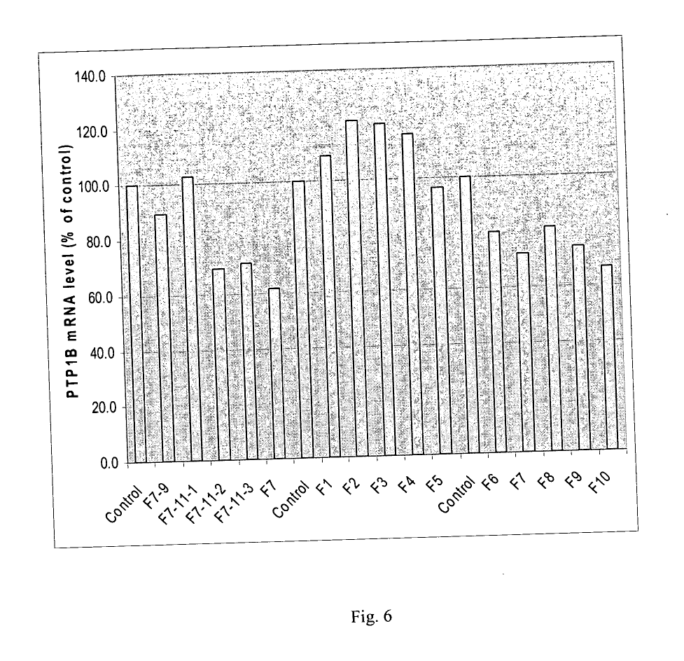

The same 10 fractions that were used to investigate ALR-2 and PEPCK inhibitory activities were also used for the initial evaluation of the components of an extract of Artemisia dracunculus as potential inhibitors of PTP-1B activity. Fractions 7, 8, 9 and 10 all exhibited PTP-1B inhibitory activity as shown in FIG. 5A. Subfractionation of fraction 7 yielded 5 active subfractions referred to as 7-4, 7-5, 7-7, 7-8, 7-9 shown in FIG. 5B. The activity of the 10 fractions of PMI-5011 to decrease PTP-1B mRNA levels is shown in FIG. 6. Fraction 7 is characterized as one of the fractions that is most potent as leading to a decrease in PTP-1B mRNA. Subfractions of fraction 7 were also active and 2′,4′-dihydroxy-4-methoxydihydrochalcone specifically reduced PTP-1B mRNA expression by 29%, suggesting that the repressed gene is involved in the inhibitory effect on PTP-1B activity. Additional purification of the subfractions of 7 enabled the identification of the active in subfraction 7-5 as Compound 6, subfraction 7-7 as Compound 5 and subfraction7-9 as Compound 4. Compound 4 in subfraction 7-9 was identified as active for ALR-2 and PEPCK inhibition as well. The other fractions or sub-fractions with lower PTP-1B inhibitory activity were not further characterized even though they may contain compounds that have high activity, but present in low concentrations.

### Identified Compounds Contributing to Anti-Diabetic Activity of an Extract of Artemisia

### EXAMPLE 10

### 5,4-di-O-caffeoylquinic acid (Compound 1)

This compound had the highest inhibitory activity against ALR2 compared to the other three compounds of davidigenin, 6-demethoxycapillarisin, and 2′,4′-dihydroxy-4-methoxydihydrochalcone isolated from Artemisia dracunculus or to the positive control, quercitrin, as shown in FIG. 1.

### Davidigenin (Compound 2)

One of the active compounds in F7-2, shown in FIG. 1D, was identified as davidigenin. This compound shows ALR2 inhibition activity.

### 6-demethoxycapillarisin (Compound 3)

A second purified compound in F7-2 was identified as 6-demethoxycapillarisin, a naturally occurring 2-phenoxychromone. This compound shows ALR2 inhibition activity.

### 2′,4′-dihydroxy-4-methoxydihydrochalcone (Compound 4)

This compound isolated from Artemisia dracunculus inhibits the activity of ALR2 and PTP-1B. This compound was also shown to decrease the gene expression of PEPCK in hepatoma cell cultures.

### 2′,4-dihydroxy-4′-methoxydihydrochalcone (Compound 5)

The identity of this dihydrochalcone was confirmed by NMR. This compound was shown as an inhibitor of PTP-1B.

### Sakurantin (Compound 6)

Sakuranetin was identified by GC-MS spectral matching to a searchable library and an authentic chemical standard with confirmation by NMR. This compound was shown to decrease PTP-1B activity as a mode of action to enhance insulin sensitivity.

### EXAMPLE 11

### Extract Pharmaceutical Compositions and Administration

Therapeutic pharmaceutical compositions are within the scope of the present invention. Such pharmaceutical compositions may comprise an effective dose of a plant extract such as a mildly polar extract of Artemisia dracunculus, in admixture with a pharmaceutically or physiologically acceptable formulation agent selected for suitability with the mode of administration. Exemplary pharmaceutical compositions may comprise an effective dose of one or more plant extracts such as one or more mildly polar extracts of Artemisia dracunculus or compound thereof, in admixture with a pharmaceutically or physiologically acceptable formulation agent selected for suitability with the mode of administration. Acceptable formulation materials preferably are nontoxic to recipients at the dosages and concentrations employed.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen sulfite); buffers (such as borate, bicarbonate, Tris-HCI, citrates, phosphates, other organic acids and salts thereof); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides and other carbohydrates (such as glucose, mannose, or dextrins); proteins (such as serum albumin, gelatin or immunoglobulins); coloring; flavoring and/or diluting agents; emulsifying agents; hydrophilic polymers (such as polyvinylpyrrolidone); low molecular weight polypeptides; salt-forming counterions [such as alkali metal ions, alkaline earth metal ions, halogen ions, organic cations, organic ions, complex ions and any other counterion known in the art (preferably sodium)]; preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, or polysorbates such as polysorbate 20, polysorbate 80, triton, tromethamine, lecithin, cholesterol, tyloxapal); stability enhancing agents (sucrose or sorbitol); tonicity enhancing agents [such as alkali metal halides (preferably sodium or potassium chloride), mannitol, or sorbitol]; delivery vehicles; diluents; excipients and/or pharmaceutical adjuvants. (Remington's Pharmaceutical Sciences, 18th Edition, A. R. Gennaro, ed., Mack Publishing Company, 1 990).

The optimal pharmaceutical composition is determined by one skilled in the art depending upon, for example, the intended route of administration, delivery format, and desired dosage. See for example, Remington's Pharmaceutical Sciences. Such compositions may influence the physical state, stability, rate of in vivo release, and rate of in vivo clearance of the plant extracts, such as the mildly polar extracts of plants such as Artemisia dracunculus.

The primary vehicle or carrier in a pharmaceutical composition is either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefor. In some embodiments of the present invention, mildly polar plant extract compositions may be prepared for storage by mixing the selected composition having the desired degree of purity with optional formulation agents (Remington's Pharmaceutical Sciences) in the form of a lyophilized cake or an aqueous solution. Further, the extract product may be formulated as a lyophilizate using appropriate excipients such as sucrose.

The pharmaceutical compositions of the mildly polar extracts may be selected for parenteral delivery. Alternatively, the compositions may be selected for delivery through the respiratory tract or digestive tract, such as orally or through a nasogastric tube. The preparation of such pharmaceutically acceptable compositions is within the skill of the art.

The formulation components are present in concentrations that are acceptable to the site of administration. For example, buffers are used to maintain the composition at physiological pH or at slightly lower pH, typically within a pH range of about 5 to about 8.

When parenteral administration is contemplated, the therapeutic compositions for use in this invention may be in the form of a pyrogen-free, parenterally acceptable aqueous solution comprising the desired plant extract in a pharmaceutically acceptable vehicle. A particularly suitable vehicle for parenteral injection is sterile distilled water in which the extract is formulated as a sterile, isotonic solution, properly preserved. Yet another preparation may involve the formulation of the desired molecule with an agent, such as injectable microspheres, bio-erodable particles, polymeric compounds (polylactic acid, polyglycolic acid), beads, or liposomes, which provides for the controlled and/or sustained release of the product which may then be delivered via a depot injection. Hyaluronic acid may also be used, and this may have the effect of promoting sustained duration in the circulation. Other suitable means for the introduction of the desired molecule include implantable drug delivery devices.

As noted above, it is contemplated that certain formulations may be administered orally. In some embodiments of the present invention, mildly polar extracts of a plant such as Artemisia dracunculus may be formulated for oral delivery with or without those carriers customarily used in the compounding of solid dosage forms such as tablets and capsules. For example, a capsule may be designed to release the active portion of the formulation at the point in the gastrointestinal tract where bioavailability is maximized and pre-systemic degradation is minimized. Additional agents may be included to facilitate absorption of the mildly polar plant extracts. Diluents, flavorings, low melting point waxes, vegetable oils, lubricants, suspending agents, tablet disintegrating agents, and binders may also be employed.

Another pharmaceutical composition may involve an effective quantity of an extract of a plant such as Artemisia dracunculus in a mixture with a non-toxic excipient which is suitable for the manufacture of tablets. By dissolving the tablets in sterile water, or other appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving extracts of plants such as Artemisia dracunculus in sustained- or controlled-delivery formulations. Products for formulating a variety of other sustained- or controlled-delivery compositions include liposome carriers, bio-erodable microparticles or porous beads and depot injections, and others, all of which are known to those skilled in the art. See for example, PCT/US93/00829, which describes controlled release of porous polymeric microparticles for the delivery of pharmaceutical compositions. Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919, EP 58,481), copolymers of L-glutamic acid and gamma ethyl-L-glutamate (Sidman et al., Biopolymers, 22:547-556, 1983), poly (2-hydroxyethyl-methacrylate) (Langer et al., J. Biomed. Mater. Res., 15:167-277, 1981) and Langer et al., Chem. Tech., 12:98-105, 1982), ethylene vinyl acetate (Langer et al., supra) or poly-D(−)-3-hydroxybutyric acid (EP 133,988). Liposomes may be prepared by any of several methods known in the art. See, e.g., Eppstein et al., Proc. Natl. Acad. Sci. USA, 82:3688-3692, 1985; EP 36,676; EP 88,046; EP 143,949.

The pharmaceutical composition of an extract of a plant such as Artemisia dracunculus to be used for in vivo administration typically must be sterile. This may be accomplished by filtration through sterile filtration membranes. Where the composition is lyophilized, sterilization using this method may be conducted either prior to, or following, lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

### RESULTS

The present invention provides a method for isolating compounds from the ethanolic extract of Artemisia dracunculus that are novel to this species and have ALR2 inhibitory activity that is similar to or greater than quercitrin, a well-known ALR2 inhibitor. The four compounds of 4,5-Di-O-caffeoylquinic acid, davidigenin, 6-demethoxycapillarisin and 2′,4′-dihydroxy-4-methoxydihydrochalcone are novel identified ALR2 inhibitors. The data shows that the extract contains additional sub-fractions with significant ALR2 inhibitory activity, as shown in FIGS. 1A-1F. The crude alcoholic extract is only slightly less active than the pure quercitrin positive control, and each of the purified compounds is only similar or slightly more active than quercitrin.

The present invention provides a novel, Real-Time PCR-based assay to guide the fractionation and isolation of the compounds that decrease PEPCK expression based on the inhibition of dexamethasone-stimulated PEPCK mRNA expression in H4IIE hepatoma cell line using insulin as a positive control. Two compounds were purified by preparatory HPLC and identified by LC-MS, 1H-, C13- and 2D NMR as 6-demethoxycapillarisin and 2′,4′-dihydroxy-4-methoxydihydrochalcone. They were able to reduce PEPCK mRNA levels by 68% at 10 μg/ml and more than 50% at 25 μg/ml, respectively.

The bioactive components of an extract of Artemisia dracunculus of the present invention was also assessed in primary skeletal muscle culture from subjects with type 2 diabetes with use of protein tyrosine phosphatase-1B (PTP-1B) activity. Of the ten fractions prepared, fractions 7-10 decreased PTP-1B activity by 20-41% in muscle cell cultures. Fraction 7 was identified as the most active fraction to inhibit PTP-1B activity and promote glucose uptake so additional preparatory HPLC methods were used to obtain subfractions of Fraction 7. Three distinct compounds were identified in sub-fractions of Fraction 7 having PTP-1B inhibitory activity and were purified from the extract. They were identified as 2′,4′-dihydroxy-4-methoxydihydrochalcone, 2′,4-dihydroxy-4′-methoxydihydrochalcone and sakuranetin and had PTP-1B inhibitory activities of as high as 26% 36% and 50% respectively. The dihydrochalcone compounds are constitutional isomers of each other but not predicted to share the same activity as they have different polarities. While they do both exhibit PTP-1B inhibitory activity, only 2′,4′-dihydroxy-4-methoxydihydrochalcone also has ALR2 inhibitory activity.

The change in PTP-1B activity associated with treatment of skeletal muscle cell cultures may be the result of either a physical change in the activity of the enzyme or to a change in the concentration of the enzyme present within the cells. The effects of the extract of Artemisia, its fractions and compounds purified from it were therefore examined with respect to the expression of the gene coding for PTP-1B by measuring the amount of PTP-1B mRNA expression in treated skeletal muscle cells. The effects of the components of the extract on mRNA levels of PTP-1B correspond to their effects on PTP-1B activity, as shown in FIG. 6. The activity of the inhibitors of PTP-1B activity and MRNA expression are not proportionately greater than the activity of the crude extract of Aritemisia dracunculus suggesting that the overall activity of the extract is dependent upon more than the additive effects of the identified compounds of 2′,4′-dihydroxy-4-methoxydihydrochalcone, 2′,4-dihydroxy-4′-methoxydihydrochalcone and sakuranetin.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

1-28. (canceled)

29. A pharmaceutical composition comprising one or more isolated compounds selected from the group consisting of 4,5-Di-O-caffeoylquinic acid, 2′,4′-dihydroxy-4-methoxydihydrochalcone, and 2′,4-dihydroxy-4′-methoxydihydrochalcone, wherein said composition is formulated for internal administration.

30. (canceled)

31. A purified composition comprising two or more isolated compounds selected from the group consisting of 4,5-Di-O-caffeoylquinic acid, davidigenin, 6-demethoxycapillarisin, 2′,4′-dihydroxy-4-methoxydihydrochalcone, 2′,4-dihydroxy-4′-methoxydihydrochalcone and sakuranetin wherein said one or more compounds are derived from a plant or plant extract of Artemisia dracunculus.

32. (canceled)

33. The composition of claim 31, further comprising at least one additive.

34. The composition of claim 33, wherein said additive is a pharmaceutically acceptable carrier, excipient, diluent or solvent

35. The composition of claim 33, wherein said composition is formulated as a capsule, tablet, syrup, concentrate, powder, granules, aerosol, or bead.

36. A composition for modulating enzyme aldose reductase (ALR2) activity in a mammal comprising isolated 4,5-Di-O-caffeoylquinic acid, and 2′,4′-dihydroxy-4-methoxydihydrochalcone.

37. A composition for modulating protein tyrosine phosphatase-1B (PTP-1B) activity in a mammal comprising two or more isolated compounds selected from 2′,4′-dihydroxy-4-methoxydihydrochalcone, 2′,4-dihydroxy-4′-methoxydihydrochalcone and sakuranetin.

38. A composition for modulating hepatic glucose output in a mammal comprising the isolated compounds 6-demethoxycapillarisin and 2′,4′-dihydroxy-4-methoxydihydrochalcone.

39. The pharmaceutical composition of claim 1 further comprising the isolated compound davidigenin.

40. The pharmaceutical composition of claim 1 further comprising the isolated compound 6-demethoxycapillarisin.

41. A pharmaceutical composition comprising three or more isolated compounds selected from the group consisting of 4,5-Di-O-caffeoylquinic acid, davidigenin, 6-demethoxycapillarisin, 2′,4′-dihydroxy-4-methoxydihydrochalcone, and 2′,4-dihydroxy-4′-methoxydihydrochalcone.

42. A pharmaceutical composition comprising two or more isolated compounds selected from the group consisting of 4,5-Di-O-caffeoylquinic acid, 6-demethoxycapillarisin, 2′,4′-dihydroxy-4-methoxydihydrochalcone, and 2′,4-dihydroxy-4′-methoxydihydrochalcone.

43. A pharmaceutical composition comprising two or more isolated compounds selected from the group consisting of 4,5-Di-O-caffeoylquinic acid, davidigenin, 2′,4′-dihydroxy-4-methoxydihydrochalcone, and 2′,4-dihydroxy-4′-methoxydihydrochalcone.

44. The composition of claim 36, further comprising davidigenin.

45. The composition of claim 36, further comprising 6-demethoxycapillarisin.
